(19) United States
(12) Patent Application Publication
(10) Pub. No.: US 2018/0153451 A1
(43) Pub. Date: Jun. 7, 2018

(54) SWEAT INDICATION OF PHYSIOLOGICAL STATES

(71) Applicant: Eccrine Systems, Inc., Cincinnati, OH (US)

(72) Inventors: Jason Heikenfeld, Cincinnati, OH (US); Robert Beech, Cincinnati, OH (US); Damon Pendarvis, Cincinnati, OH (US); Gavi Begtrup, (US)

(73) Assignee: Eccrine Systems, Inc., Cincinnati, OH (US)

(21) Appl. No.: 15/576,106

(22) PCT Filed: Jun. 6, 2016
(86) PCT No.: PCT/US16/36038

§ 371 Date: Nov. 21, 2017

Related U.S. Application Data
(60) Provisional application No. 62/171,578, filed on Jun. 5, 2015.
(60) Provisional application No. 62/327,408, filed on Apr. 25, 2016.

(51) Int. Cl. A61B 5/145; A61B 5/01; A61B 5/00; A61B 5/024; A61B 5/11; A61B 10/00; A61B 5/1495; G01N 33/487

(52) U.S. Cl. CPC A61B 5/14517; A61B 2562/028; A61B 5/14539; A61B 5/14546; A61B 5/4266; A61B 5/4875; A61B 5/7246; A61B 5/024; A61B 5/1118; A61B 5/4845; A61B 10/0012; A61B 5/1495; A61B 5/6833; A61B 10/0064; G01N 33/48792; A61B 5/14532; A61B 2560/0252; A61B 2562/029; A61B 2562/0271; A61B 5/01

(57) ABSTRACT

The disclosed invention includes: a device and method of performing physiological sweat sensing device calibration; a device and method of indicating an individual's dehydration state; a device and method of indicating an individual's stage in their ovulation cycle; a device and method of indicating that an individual is hypokalemic or hyperkalemic; a device and method of indicating that an individual is entering a hypoglycemic state; a device and method of indicating an individual's glucose trend value; and a device and method of indicating that an individual has experienced toxic substance exposure.

### CROSS-REFERENCE TO RELATED APPLICATIONS

The present application has specification that builds upon U.S. Provisional Application No. 62/171,578, filed Jun. 5, 2015, and U.S. Provisional Application No. 62/327,408, filed Apr. 25, 2016, the disclosures of which are hereby incorporated herein by reference in their entirety.

### BACKGROUND OF THE INVENTION

Sweat sensing technologies have enormous potential for applications ranging from athletics, to neonates, to pharmacological monitoring, to personal digital health, to name a few applications. This is because sweat contains many of the same biomarkers, chemicals, or solutes that are carried in blood, which can provide significant information which enables one to diagnose ailments, health status, toxins, performance, and other physiological attributes even in advance of any physical sign. Furthermore, sweat itself, and the action of sweating, or other parameters, attributes, solutes, or features on or near skin or beneath the skin, can be measured to further reveal physiological information.

In particular, sweat sensors hold tremendous promise for use in workplace safety, athletic, military, and clinical diagnostic settings. A primary goal of the present invention is to provide decision support to a sweat sensing device user that is informative at the level of the individual patient. A sweat sensing patch worn on the skin and connected to a computer network via a reader device, such as a smart phone or other portable or stationary device, could aid in recognition of the physiological state of an individual, and relay crucial data about dehydration levels, physiological stress levels, ovulation cycle or other physiological states. In certain settings, sweat sensors may continuously monitor certain aspects of an individual's physiological state and communicate relevant information to a reader device or computer network, which would then compare collected data to threshold readings and generate notification messages to the individual, a caregiver, a work supervisor, or other device user.

For example, an individual's dehydration state could be indicated by continuously monitoring for dehydration biomarkers as they present in sweat. Similarly, an individual's stage in their ovulation cycle may be monitored through certain hormone levels in sweat. Whether an individual is experiencing a hypoglycemic state can also be indicated through monitoring the relationship of sweat glucose, cortisol, and sweat rate. Methods to derive these physiological states and others are contemplated within the scope of the present invention.

Before continuing with the background, a variety of definitions should be made, these definitions gaining further appreciation and scope in the detailed description and embodiments of the present invention.

Sweat sensor data means all of the information collected by sweat system sensor(s) and communicated via the system to a user or a data aggregation location.

Correlated aggregated sweat sensor data means sweat sensor data that has been collected in a data aggregation location and correlated with outside information such as time, temperature, weather, location, user profile, other sweat sensor data, or any other relevant data.

Hardware calibration means the correction of sweat sensing device outputs based on the mechanical environment of the device measurement. For example, a correction based on output differences resulting from a particular sensor or batch of sensors, or other factors, would be considered to receive Hardware calibration. Such calibration can be manual, meaning a device user must choose an appropriate correction factor for each sensor; or automatic, meaning a device performs at least a portion of the calibration process without input from the device user.

Measurement values means sensor output translated to a value appropriate to the sensor, e.g. volts, ohms, ° C., mMol, siemens, etc.

Biological or Bio calibration means the correction of sweat sensing device outputs based on the physiological environment of the device measurement. For example, outputs corrected by pH; temperature; sweat rate; sweat salinity; sweat sample concentration; sweat analyte partitioning; device wearer characteristics, such as age, body mass index, time since the wearer's last meal; or other factors, would have received Bio calibration.

First order inputs and calibration mean the use of inputs to correct or calibrate sweat sensing device outputs. For example, pH and sensor temperature inputs used to refine analyte concentration values would become first order inputs, and would produce first order calibrated outputs.

Second order inputs and calibration mean the use of inputs that have been corrected by first order calibration procedures to further calibrate or refine sweat sensing device outputs. For example, a physiological range filter constructed with pH and sensor temperature inputs would become a second order input, and would result in a second order calibration of analyte concentration values.

Galvanic skin response (GSR) means measurements of the electrical conductivity of the skin. GSR serves as a means of estimating sweat rate, since skin conductivity is dominated by the contribution of sweat, and increases linearly with increases in sweat rate throughout the linear range of 0.4 μL/cm²/min to 1.5 μL/cm²/min.

Sweat conductivity means measurements of the electrical conductivity of sweat. Sweat conductivity serves as a means of estimating Cl− content, since Cl− represents the dominant anion in sweat. However, conductivity does not precisely correlate to Cl− levels, because lactate and bicarbonate also make significant contributions to sweat conductivity. The sweat sensing device would measure sweat conductivity by means of an electrode.

Sensor response lag means the difference between the point in time that a sweat sample reaches an ionophore sensor and the point at which the sensor produces an electrical signal corresponding to the sweat sample.

This has served as a background for the present invention, including background technical invention needed to fully appreciate the present invention, which will now be summarized.

### SUMMARY OF THE INVENTION

The present disclosure is premised on the realization that sweat can be effectively analyzed in a single, continuous, or repeated manner inside the same device, and addresses applications of a sweat sensing device based on such capabilities. Specifically, the disclosure provides: a device and method of performing physiological sweat sensing device calibration; a device and method of indicating an individual's dehydration state; a device and method of indicating an individual's stage in their ovulation cycle; a device and method of indicating that an individual is hypokalemic or hyperkalemic; a device and method of indicating that an individual is entering a hypoglycemic state; a device and method of indicating an individual's glucose trend value; and a device and method of indicating that an individual has experienced toxic substance exposure.

### BRIEF DESCRIPTION OF THE DRAWINGS

The objects and advantages of the present invention will be further appreciated in light of the following detailed descriptions and drawings in which:

FIG. 1 is an example sweat sensing device of the present disclosure.

FIG. 2 is a more detailed depiction of a sweat sensing device of the present disclosure.

FIG. 3 is an example flow chart representing a method of performing physiological calibration.

FIG. 4 is a chart depicting the relationship between sweat ion concentrations and sweat generation rate.

FIG. 5 is a chart depicting a linear relationship between sweat Na+ concentration and sweat generation rate.

FIG. 6 is a chart depicting the relationship between GSR and sweat generation rate.

FIG. 7 is a chart depicting the relationship between GSR, sweat Na+ concentration, and sweat generation rate.

FIG. 8 is a depiction of a sweat sensing device of the present disclosure modeling the mixing of old and new sweat in a sweat volume.

FIG. 9 is a chart depicting the integration of sweat Na+ concentration area to correct Na+ loss calculations during periods of declining sweat rate.

FIG. 10 is an example flow chart representing a method of indicating an individual's dehydration state.

FIG. 11 is an example flow chart representing a method of indicating an individual's local muscle group lactate measurement.

FIG. 12 is an example flow chart representing a method of indicating an individual's stage in their ovulation cycle.

FIG. 13 is an example flow chart representing a method of indicating that an individual is entering a hypoglycemic state.

FIG. 14 is an example flow chart representing a method of indicating an individual's glucose change rate.

### DETAILED DESCRIPTION OF THE INVENTION

The detailed description of the present invention will be primarily, but not entirely, limited to devices, methods and sub-methods using wearable sweat sensing devices. Therefore, although not described in detail here, other essential steps which are readily interpreted from or incorporated along with the present invention shall be included as part of the disclosed invention. The disclosure provides specific examples to portray inventive steps, but which will not necessarily cover all possible embodiments commonly known to those skilled in the art. For example, the specific invention will not necessarily include all obvious features needed for operation. Several specific, but non-limiting, examples can be provided as follows. The invention includes reference to the article in press for publication in the journal IEEE Transactions on Biomedical Engineering, titled “Adhesive RFID Sensor Patch for Monitoring of Sweat Electrolytes”; the article published in the journal AIP Biomicrofluidics, 9 031301 (2015), titled “The Microfluidics of the Eccrine Sweat Gland, Including Biomarker Partitioning, Transport, and Biosensing Implications”; as well as U.S. Provisional Application No. 62/064,009, and U.S. Provisional Application No. 62/120,342; each of which is included herein by reference in their entirety.

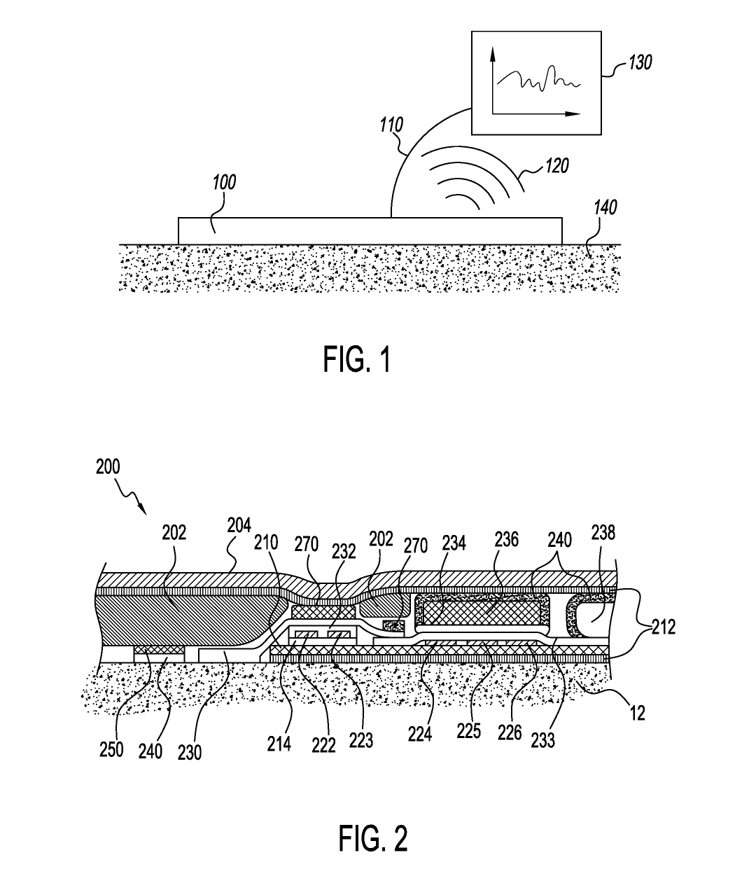

With reference to FIG. 1, a representative sweat sensing device 100 to which the present disclosure applies is placed on or near skin 12. The sweat sensing device may be fluidically connected to skin or regions near skin through microfluidics or other suitable techniques. Device 100 is in wired communication 152 or wireless communication 154 with a reader device 150, which could be a smart phone or portable electronic device, or for some devices, the device 100 and reader device 150 can be combined. Communication 152 or 154 is not constant and could be a simple one-time data download from device 100 once it has completed its measurements of sweat.

With reference to FIG. 2, a more detailed partial view of a sweat sensing device is provided as device 200. The device 200 may include the following elements: a filler material 202; a textile covering 204; a substrate 210; adhesive 212; a self-leveling material 214; a wicking volume reducing component 230; a sweat stimulant gel composed of sweat stimulant and agar 240; and an iontophoresis electrode 250.

The device further includes at least one sensor(s) 222, 223; an optional hydrogel 232 for enhancing fluidic contact between substrate 210 and a rigid component 270 such that the wicking volume reducing component 230 and the at least one sensor 222, 223 are in fluidic contact at all times. The device further includes a wicking or microfluidic component 233; at least one electrochemical aptamer sensor(s) 224, 225, 226; a forward osmosis membrane 234; a polymer seal 214; an osmosis pumping material 236; and a wicking pumping material 238.

With further reference to FIG. 2, the device could operate as follows: electrode 250, and gel 240 provide iontophoretic sweat stimulation as needed. Once sweat is stimulated, electrode 250 can also be used to measure skin impedance, which can be used to derive a proportional measure of electroporation and/or sweat generation rate. Wicking volume reducing component 230 then wicks stimulated sweat off the skin surface and carries the sweat sample to at least one sensor(s) 222, 223, which would measure Na+, K+, or Cl−. Wicking component 230 may also include a thermal flow meter to measure sweat rate. Wicking component 233 then transports the sweat sample onto electrochemical aptamer sensors 224, 225, 226. The analytes will be concentrated as water and small sweat solutes are transported through the forward osmosis membrane 234 and out of the sweat sample. Finally, wicking material 238 absorbs the sweat sample, and at least partially drives sweat sample flow through the device.

As disclosed, a sweat sensing device might include a plurality of sensors to improve detection of sweat analytes, including a reference electrode, a pH sensor, a temperature sensor, a galvanic skin response sensor, a sweat conductivity sensor, a skin impedance sensor, a capacitive skin proximity sensor, and an accelerometer. Many of the auxiliary features of the invention may, or may not, require other aspects of a sweat sensing device, including two or more counter electrodes, reference electrodes, or additional supporting technology or features, which are not captured in the description herein, such as an onboard real-time clock, onboard flash memory (i.e. 1 MB minimum), Bluetooth™ or other communications hardware, and a multiplexer to process a plurality of sensor outputs.

The disclosed sweat sensing device also includes computing and data storage capability sufficient to operate the device, which incorporates the ability to conduct communication among system components, to perform data aggregation, and to execute algorithms capable of generating notification messages. The device may have varying degrees of onboard computing capability (i.e., processing and data storage capacity). For example, all computing resources could be located onboard the device, or some computing resources could be located on a disposable portion of the device and additional processing capability located on a reusable portion of the device. Alternatively, the device may rely on portable, fixed or cloud-based computing resources.

The sweat sensing device's data aggregation capability may include collecting all of the sweat sensor data generated by sweat sensing devices and communicated to the device. The aggregated sweat sensor data could be de-identified from individual wearers, or could remain associated with an individual wearer. Such data can also be correlated with outside information, such as the time, date, medications, drug sensitivity, medical condition, activity performed by the individual, motion level, fitness level, mental and physical performance during the data collection, body orientation, the proximity to significant health events or stressors, age, sex, health history, or other relevant information. The reader device or companion transceiver can also be configured to correlate speed, location, environmental temperature or other relevant data with the sweat sensor data. The data collected could be made accessible via secure website portal to allow sweat system users to perform safety, compliance and/or care monitoring of target individuals. The sweat sensor data monitored by the user includes real-time data, trend data, or may also include aggregated sweat sensor data drawn from the system database and correlated to a particular user, a user profile (such as age, sex or fitness level), weather condition, activity, combined analyte profile, or other relevant metric. Trend data, such as a target individual's hydration level over time, could be used to predict future performance, or the likelihood of an impending physiological event. Such predictive capability can be enhanced by using correlated aggregated data, which would allow the user to compare an individual's historical analyte and external data profiles to a real-time situation as it progresses, or even to compare thousands of similar analyte and external data profiles from other individuals to the real-time situation. Sweat sensor data may also be used to identify wearers that are in need of additional monitoring or instruction, such as the need to drink additional water, or to adhere to a drug regimen.

Because the sweat sensing device could produce potentially sensitive physiological data, some database fields will be routinely encrypted. A preferred encryption method is the Advanced Encryption Standard. The device will access a random 128 bit encryption and decryption key that will be generated and stored by a companion reader device when needed for data transmission. In addition, because some sweat sensor data may repeat frequently, additional protection will be provided by introducing a random initialization vector before the encryption of each value. This will prevent observable patterns from emerging in the encrypted sweat sensor data. Other encryption methods and steps may be required and will be applied according to best practices, as known to those skilled in the art.

Sweat is known to contain a large number of compounds that could be used to indicate an individual's physiological state. In general, determining an individual's physiological state is a significant challenge. Not only is every individual different in terms of how a physiological state may present, but even a simple physiological state or disorder is a complex set of biological processes that does not readily lend itself to reduction. Consequently, a definitive diagnosis of a physiological condition often is not possible. Rather, it becomes necessary to divide individuals according to phenotypes or susceptibilities that indicate the mode in which a physiological state is likely to manifest in those individuals. These phenotypes may be indicated by analyte signatures that emerge in sweat. Among the most common substances found in sweat are the following: Na+, Cl−, K+, Ammonium (NH4+), urea, lactate, glucose, serine, glycerol, cortisol, and pyruvate. In addition to these common sweat analytes, each physiological condition may also have particular sweat analytes that will prove informative for indicating that physiological state. For example, blood creatinine levels have proven useful for indicating hydration levels, which also may prove true for sweat.

To date, there have been few studies linking sweat analytes to physiological states. Among these are studies linking increased sweat chloride levels with cystic fibrosis, or a spike in chloride levels with ovulation. It will likely be necessary to build data across multiple individuals correlating physiological states with sweat sensor readings. By this means, certain phenotypes can be identified for which a given physiological state will manifest in a discernible sweat analyte signature.

Further, translation of analyte concentrations and ratios to meaningful physiological information will have to account for a number of variabilities unrelated to differences in concentrations. For example, sweat concentrations of analytes relative to blood or plasma concentrations are known to vary depending on sweat rate, the body location from which a sample is taken, kidney or liver disease or function, external temperatures, and other factors. To develop meaningful physiological information, it will therefore be necessary to develop algorithms and techniques that reflect how the various analyte signatures change in response to these variabilities.

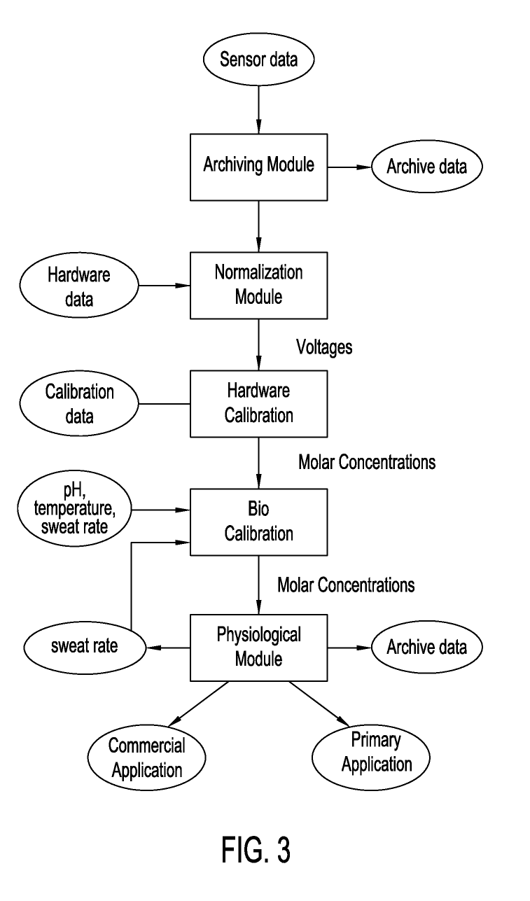

With reference to FIG. 3, aspects of the present disclosure represent a series of functions or modules that facilitate sweat sensing device operation. These different modules represent the process of transforming raw sweat sensor data into meaningful physiological information about a sweat sensing device wearer. In the Archive Module, raw data is stored prior to algorithm processing. In the Normalization Module, the algorithm first transforms the electrical output from a device sensor, e.g., an ionophore analyte sensor, or a pH sensor, into voltage values. The Calibration Module applies a hardware correction for sensor variability and sensor function, and transforms the voltages into physiologically relevant values, i.e., molar concentration for sweat analytes, or pH for H+ concentration. The Calibration Module also includes a biological calibration step that corrects the sweat sensor data according to the presence of sweat, sweat sample pH, sensor temperature, measured sweat rate, and other factors. The calibrated concentration data is then passed to a Physiological Module, which transforms measured and calibrated sensor outputs into new physiologically informative data, such as calculated sweat rate data, dehydration status, or a glucose trend. The device may further refine the sensor outputs by passing the new data back to the Calibration Module to apply additional corrections before finally sending the refined data to a quality control archive, a commercial health data application and/or a Primary Application.

The Archiving Module stores raw electrical signal data from the sweat sensing device to allow future use of the data. The Archiving Module takes the data produced by an onboard sensor and stores it in a database, such as the Apple Core Data™ database, or other storage means located onboard a companion reader device. Archived data could be stored in numerous formats as known in the art, including comma separated value (“CSV”) files, or a database, and will include information associating the data with the originating sensor, and other relevant characteristics of the device or measurement. For example, the data fields associated with each archived sweat sensor measurement may comprise the date and time the measurement was taken, the data source (i.e. type of device or Physiological Module), the unique data source identifier, the unique sensor identifier, type of measurement (i.e., Cl−, temperature, GSR, pH, sweat conductivity), measurement units (e.g., volts, ohms, ° C., mMol, siemens), calibration data, and the encrypted actual measured value. By automatically archiving such data before further processing, and by associating the measured data with the originating device or module that produced it as output, archived data may later be used to derive additional or improved physiological information from the data, as interpretive techniques and equipment improve with time.

With further reference to FIG. 3, the Normalization Module transforms raw electrical signals from sensors into voltage, current, or other appropriate measurement values, when not already done so by the device. The Normalization Module processes the same data as is sent to the Archiving Module. The raw electrical signal outputs could be interpreted as different measurement values depending on the type of sensor, the analyte being detected, the device electronics, signal processing, and other hardware differences. The Normalization Module accounts for these hardware differences and produces a standardized output. The algorithm accesses stored information about relevant past sweat sensors and sweat sensing devices, so that the module may process raw data from any hardware source into the appropriate measurement value. As normalization processing improves, the algorithm will be able to transform archived data from earlier generation hardware into measurement values based on the latest understanding of the translation process, which will be embodied in the algorithm.

The Normalization Module may also be performed prior to algorithmic processing. For example, a sweat sensing device or individual sensor hardware may include data storage capability that allows the component to store normalization data particular to the hardware in use. By using such onboard normalization data, the first data such a device would send into the algorithm would be in the form of, for example, voltage values. In such cases, the archived “raw data” would be in the form of voltages, and the Normalization Module would simply pass the voltage values through without additional processing. In other cases, the device may pass on current, concentration, temperature, or other measurement values, as appropriate.

With further reference to FIG. 3, the Calibration Module transforms measurement values from the Normalization Module into physiologically appropriate units, such as molar concentration values for a sweat analyte measurement, or degrees Celsius for a temperature measurement. To perform translations of data values into meaningful information, the calibration function must first acquire Hardware Calibration to account for variances in individual sensor hardware or differences in sensor performance. Next, it performs Bio Calibration to adjust the data for multiple physiological and environmental factors that may affect the voltage readings. This module may employ an iterative process with a plurality of nested subroutines to allow the output to account for various inputs and produce successively refined, and hence more accurate, outputs.

Hardware Calibration is aimed at accounting for variances in individual sensor outputs and performance, and can be accomplished manually or automatically. Manual Hardware Calibration describes a process in which a device user selects an analyte to be detected, and inputs the appropriate concentration(s) of one or more calibration solutions. The device then performs measurements in the one or more calibration solutions and calculates a translation factor based on the difference between the actual measurements and the known concentrations. In a preferred embodiment, individual sensors could be configured to perform automatic Hardware Calibration. After sweat sensing device assembly, individual sensors are calibrated by placing the sensor in one or more calibration solutions of known molarity for the target analyte. The device would store the calibration measurements in memory as calibration samples. The algorithm would not necessarily limit the number of calibration samples that may be taken by each sensor, and will adjust analyte measurements based on the most recent calibration sample value(s). When the device is activated on a wearer's skin, and the sensors measure analytes in sweat, the device compares the individual sensor readings to the stored calibration sample values. The detected measurements are then adjusted based on the calibration values. The device passes the calibrated data to the Archive Module and to the Normalization Module. The calibrated data will pass through modules unchanged if the respective transformations have already been accomplished earlier in the process. Automatic calibration may also be accomplished on lots or other subsets of sensors, and during use, the subset would be calibrated to a translation factor calculated for that particular group of sensors.

The device may also perform authentication functions as part of Hardware Calibration. For example, the device may store calibration values on a microprocessor configured on the sensor tab component. Such a microprocessor may then be configured with encryption or other means to authenticate the sensor tab and device.

Biological Calibration is aimed at accounting for physiological factors that affect the meaning of the sensor outputs. Bio Calibration must account for first order inputs and second order inputs. First order inputs are those that can be measured or applied directly to the normalized data, for example, sensor temperature, measured sweat rate, sweat pH, or a physiological output range filter. Second order inputs are those that must be constructed from first order inputs, i.e., calculated sweat rate or a corrected range filter, and may be developed by a Physiological Module.

During first order Bio Calibration, molar concentration values for a target analyte that are produced by Hardware Calibration will be corrected by direct inputs. A number of relevant inputs may influence the interpretation of analyte concentration values. Some of these will be accomplished by sensor measurements that are located onboard the sweat sensing device. For example, sweat pH, sensor temperature, measured sweat rate, and skin-to-device proximity/contact, are data points that inform the interpretation of ionophore sensor measurements. In addition, the use of artificial sweat stimulation (with or without sweat stimulating chemicals like carbachol) may alter the interpretation of sensor measurements, and the data must accordingly be corrected. Further, the sweat partitioning model applicable to specific analytes may also be the basis for output corrections. For example, larger analytes, such as proteins and nucleotides could be released into sweat by low voltage sweat pore electroporation. The resulting sweat concentrations of the larger analytes must then be adjusted to meaningfully correlate with blood concentrations. Other first order inputs may include inputs from outside the sweat analyte sensors, including other wearable device inputs, like galvanic skin response or heart rate, blood oxygenation, or commercial health and fitness application inputs, for example from Apple HealthKit™.

A first order calibration of an analyte concentration measurement may also take the form of a physiological filtering routine. The filtering routine will compare molar concentration outputs to expected maximum and minimum values for the analyte being measured. For example, when detecting sweat Cl− concentrations, the filtering routine would compare a detected result to an expected minimum concentration value (approximately 0 mM) and an expected maximum concentration value (approximately 200 mM). If the detected Cl− concentration were, for example 450 mM, the data from that sensor would be weighted or discarded, as appropriate. The device could then initiate a diagnostic routine to troubleshoot the sensor producing bad data. If the detected concentration were within limits, the filtering routine would pass along the data unaltered, and the device could identify the source sensor as operational. The filtering routine could become a second order calibration by adjusting the maximum and minimum values based on direct inputs like pH or sensor temperature.

During second order Bio Calibration, molar concentration values for a target analyte that are produced by either a Hardware Calibration or a first order Bio Calibration will be corrected by composite inputs. For example, as discussed above, the filtering routine would become a second order (composite) calibration if the filtering values were set based on physiological inputs such as sweat pH, measured sweat rate, etc. The use of such a composite filtering routine would provide more precise and accurate boundary values for filtering the analyte concentration.

Other second order calibration inputs could be produced by a Physiological Module. For example, the context of a device wearer's physical state may inform when to take a sweat sensing device measurement, or how much to weight such measurements. For example, measurements of sweat cortisol levels should not be taken when a wearer is exercising, or the cortisol measurements taken during exercise should be appropriately corrected to reflect the influence of the activity. To determine such contextual information, the device may employ various other sensors, including accelerometers, skin temperature, or other sensors capable of providing relevant information. Additional contextual factors that could impact the validity or weighting of sweat sensor measurements include whether the wearer is resting after a period of activity, how recently the wearer awakened, how recently the wearer ate or drank, whether the wearer is stressed or calm, the duration or intensity of physical activity, and so on. By using such second order calibration inputs, the Calibration Module could ensure that sweat measurements are physiologically meaningful.

As another example, sweat rate is an important metric that can inform many analyte concentration measurements. For prolonged, continuous, or semi-continuous analyte measurement, meaningful readings can only be taken at chronologically assured sweat sampling rates or intervals to ensure measurements are taken on fresh sweat as it emerges from a device wearer's skin. Chronological assurance may rely on a calculation of sweat rate in conjunction with sweat volume to calculate a sweat refresh rate, which informs the device when a chronologically assured measurement of analytes in fresh sweat can be taken by the sensor.

In addition, for larger molecules that do not readily partition into sweat, such as proteins, sweat rate may be a critical component of a meaningful concentration measurement. There are at least two sweat rate-related issues involved in the determination of concentrations for larger molecules. First, the levels of these analytes in sweat are on the pMol scale, which is below the sensitivity levels of most wearable sensor technologies. Therefore, sweat samples may need to be concentrated in order to reach analyte molarities that are in the detectable range. To translate the measured concentrations into physiologically meaningful information, the sweat rate will be necessary to back-calculate the measurements into a pre-concentration analyte molarity. Second, because of the low partitioning rates for such molecules, higher sweat rates will carry lower concentrations of such molecules, regardless of blood concentrations. Therefore, a sweat rate value could be necessary to correlate detected sweat analyte concentrations with blood concentrations.

For these reasons, a Physiological Module for determining sweat rate may create output data for use in performing second order calibration or other applications. A calculated sweat rate can be determined by different means, including estimating sweat rate based on interpretation of sweat Na+, Cl−, and K+ ratios. In general, sweat concentrations of Na+ and Cl− increase linearly with sweat rate, while K+ tends to remain at a relatively constant molarity as sweat rate increases.

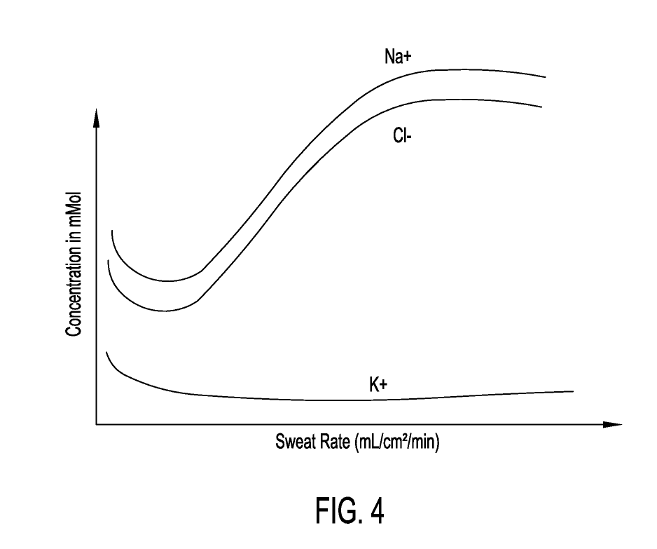

Various studies in eccrinology have characterized the variance in molarity of Na+, Cl− and K+ with sweat rate. See FIG. 4, Sato, K., et al., “Biology of sweat glands and their disorders,” J. of the Am. Academy of Dermatology, p. 552, 20/4/April 1989. Na+ and Cl− enter sweat in the secretory coil of the eccrine sweat gland, and at negligible sweat rates, are isotonic with interstitial fluid concentrations of Na+ and Cl−. Bovell, Journal of Local and Global Health Science, p. 9, 2015:5. With the initiation of sweating, Cl− is pumped into the lumen of the gland, where its negative electrical potential pulls in Na+. The Na+ and Cl− combine to form NaCl, which creates an osmotic gradient that draws water into the lumen. As the newly created sweat moves out of the secretory coil, Na+, with Cl− in tow, is reabsorbed through the duct and re-enters the interstitial fluid.

At lower sweat rates, 0.0 to 0.4 μL/cm²/min, relatively more of the Na+ and Cl− are reabsorbed by the duct so that sweat reaching the skin has lower concentrations of Na+ and Cl−. Amano, T., et al., “Determination of the maximum rate of eccrine sweat glands' ion readsorption using galvanic skin conductance to local sweat rate relationship,” Eur. J. Appl. Physiology, p. 4, DOI 10.1007/s00421-015-3275-9. Initially, between 0.2 and 0.4 μL/cm²/min sweat rate, the Na+ gland reabsorption rate is at is maximum (around 85%), which translates to Na+ concentration of 10-15 mMol. Sato, K., et al., “Biology of sweat glands and their disorders,” J. of the Am. Academy of Dermatology, p. 552, 20/4/April 1989, p. 552; Buono, M., et al., “Na+ secretion rate increases proportionally more than the Na+ reabsorption rate with increases in sweat rate,” J. Appl. Physiology, 105:1044-1048, 2008. As sweat rate increases, the amount and speed of Na+ flowing through the duct overwhelms the reabsorption mechanism, so that at sweat rates above 0.4 μL/cm²/min, the duct absorbs a significantly lower percentage of Na+, down to about 65% of Na+ at a sweat rate of 0.8 μL/cm²/min. Buono, M., et al. As a result, Na+ concentrations show a linear increase with increases in sweat rate in the range of about 20 mEq/L for a 0.4 μL/cm²/min sweat rate, to 60 mEq/L for a 1.5 μL/cm²/min sweat rate. Allen, J., et al., “Influence of acclimatization on sweat sodium concentration,” J. of Applied Physiology, 30/5/May 1971, at 710; Bovell, at 11; see also, Buono, p. 1025. (0.25 μL/cm²/min sweat rate correlated to 20 mMol/L Na+, 0.9 μL/cm²/min sweat rate correlated to 55 mMol/L Na+). Cl− concentrations roughly correspond to Na+ levels for various sweat rates, but are usually 20 mMol less. Sato, K., et al. For individuals that acclimatize to warmer environments, or who engage in physical conditioning, the body's ability to reabsorb Na+ improves, and sweat profiles for these individuals will tend to have sweat Na+ concentrations about 15 mMol lower than for unconditioned individuals. Allen, J., et al., at 710.

Similar examination of K+ molarity as it changes with sweat rate reveals an analyte that can function as a reference for a number of sweat applications, including the determination of sweat rate. Unlike Na+ and Cl−, the rate of K+ production by the sweat gland does not accelerate with sweat rate increase, and K+ is not reabsorbed by the duct in significant amounts. As a result, K+ concentration remains relatively steady throughout the range of sweat rates. At negligible sweat rates, K+ concentration in the secretory coil corresponds to plasma concentrations of about 3-4 mMol. Baker, L., et al., “Comparison of regional patch collection vs. whole body washdown for measuring sweat sodium and potassium loss during exercise,” J. Appl. Physiology, 107: 887-895, 2009. At very low sweat rates, K+ tends to enter sweat at increased levels, i.e., up to 9 mMol, before settling to a concentration of about 6 mMol. Sato, K., et al.; Bovell, at 11.

These results for Na+, Cl− and K+ molarities as they correlate to sweat rate must be caveated by acknowledging that correlations and data are less certain at low sweat rates, i.e. below 0.4 μL/cm²/min, where the Na+ production is not yet overwhelming the duct's ability to reabsorb Na+. Additionally, the linear correlation between Na+, Cl− and sweat rate may break down after extended exertion, during which the maintenance of high sweat rates with concomitant loss of electrolytes alters the level of sweat and electrolyte production by the sweat glands.

Because of these relatively predictable responses to increases in sweat rate, it is possible to determine a calculated sweat rate based on relative concentrations of Na+, Cl− and K+. For example, to determine a calculated sweat rate, the device would start with corrected Na+, Cl− and K+ molar concentration measurements produced by a sweat sensing device's sweat sensors and processed through the Normalization Module and the Calibration Module. These measurements could be hardware calibrated values, first order bio calibrated values, or second order bio calibrated values. The corrected Na+, Cl−, and K+ values would then be compared.

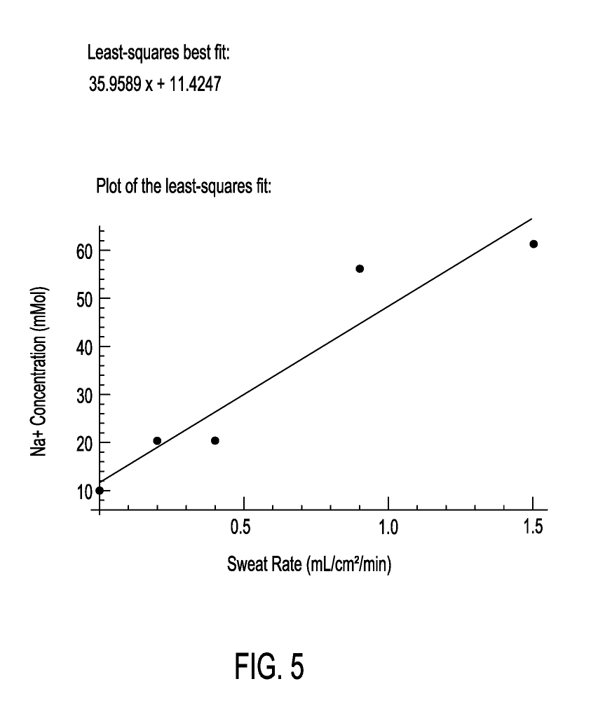

Starting with a Na+ concentration, the device could perform a rough first order calculation of sweat rate by identifying the corresponding sweat rate for that Na+ concentration from a data set, for example, a lookup table. Based on linear regression analysis of Na+ concentrations and corresponding sweat rates found in scientific literature, see FIG. 5, Na+ concentration would correlate to sweat rate along a line starting at roughly 10 mMol for 0.0 μL/cm²/min sweat rate, and having a slope of 35.9. By this method, a Na+ concentration of 25 mMol would correspond to a sweat rate of 0.42 μL/cm²/min. A lookup table could be developed for this purpose by multiple methods, varying in sophistication and customization depending on the sweat sensing device application. For example, the lookup table could be constructed using relevant data external to the sweat sensing device correlated to the particular application or individual. This data may consist of sweat sensor data, or laboratory data collected previously on the individual, data collected on individuals performing similar activities, data on individuals in similar weather conditions, data on individuals with similar phenotypical profiles, etc. In other embodiments, the device may determine sweat rate from a function or formula based on empirical correlations between Na+ concentration and sweat rate, and may account for individual, environmental, application-specific and other relevant factors.

Cl− concentrations may also be used to calculate sweat rate directly, or to calibrate Na+ measurements, and by extension sweat rate calculations. Sweat Cl− concentrations roughly mirror Na+ concentration changes with sweat rate, but are generally 20 mMol less in magnitude. Because Cl− also has a linear relationship with sweat rate, a corresponding lookup table, formula, or other calculation could be used to derive sweat rates from Cl− concentrations. Contemporaneous Cl− concentrations may also be compared to Na+ concentrations to determine if Cl− levels are appropriate relative to the measured Na+ levels. By comparison of the two values and their trends over time, the device may make a determination that one sensor is more reliable than the other. The more reliable data would then be weighted more heavily, or the bad data discarded, as appropriate. The sweat rate determined from Cl− may also be compared to the sweat rate calculated from Na+ to improve the robustness of the result.

Contemporaneous concentration measurements for K+ may also be used to calibrate the Na+ and Cl− concentrations and sweat rate. For example, at low sweat rates, K+ is known to emerge at levels above plasma concentrations, i.e. around 9 mMol, and therefore can be used to determine if the sweat sensing device is in a low sweat rate range, i.e., 0.0 to 0.4 μL/cm²/min. The device may compare Na+ and K+ measurements, for example a K+ reading of 9 mMol corresponding to a lower Na+ level, such as 15 mMol would indicate sweating in the low sweat rate range. Once the device determined that sweat rate was likely below 0.4 μL/cm²/min, the device could use a lookup table applicable to low sweat rates, or apply a corrective formula or other correction to the lookup table used for sweat rates above 0.4 μL/cm²/min.

For sweat rates above 0.4 μL/cm²/min, K+ concentration settles into a steady state of about 6 mMol. In this range, K+ concentrations may be compared to Na+ levels to determine if the wearer is sweating above 0.4 μL/cm²/min, for example a K+ reading of 6 mMol accompanied by a Na+ reading of 30 mMol would indicate a higher sweat rate. Similarly to Na+, Cl− can be used with K+ to determine if the wearer is likely in a low sweat rate range or sweating above 0.4 μL/cm²/min.

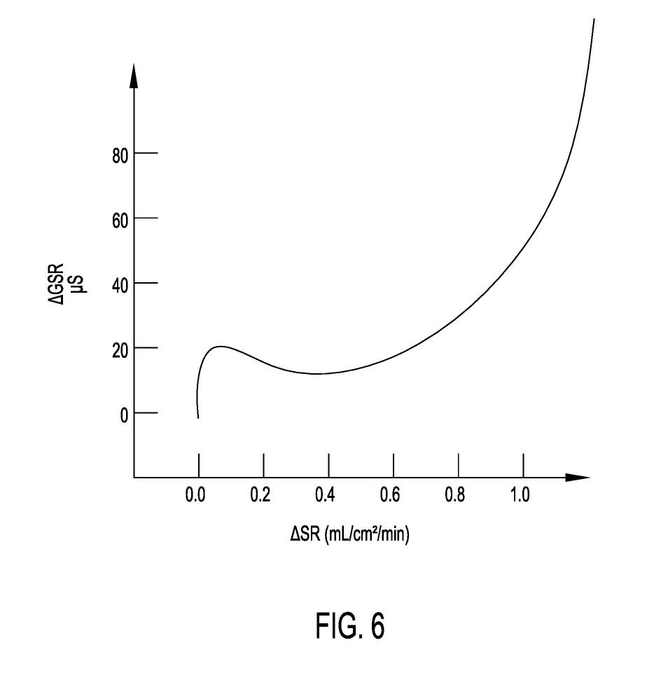

Sweat rate may also be determined from galvanic skin response (GSR) measurements that are refined by use of sensor measurements of Na+, Cl− or K+ concentrations. GSR readings taken on the skin have been shown to reflect a similar profile to that seen with Na+ and Cl− sweat concentrations. See Amano, T., et al. This is because skin conductivity as measured by GSR is dominated by the component attributable to the sweat glands, and within the sweat glands, the dominant component is sweat and its most abundant ions, Na+ and Cl−. Changes in GSR may therefore function as an independent measurement of sweat rate. With reference to FIG. 6, GSR measurements on skin indicate three phases: (1) a pre-sweating period, in which GSR increases while sweat rate remains negligible (representing the sweat duct filling with sweat); (2) a low sweat rate phase, corresponding to sweat rates below 0.4 μL/cm²/min, during which sweat rate increases while GSR stays roughly constant; and (3) a linear phase above 0.4 μL/cm²/min in which the sweat rate increases linearly with GSR. From Amano, T., et al. In some embodiments of the present disclosure, therefore, a GSR measurement may indicate whether a device wearer is in one of three sweating phases, i.e., about to sweat, sweating at a low sweat rate, or sweating in the linear range.

Additionally, GSR can be used to approximate NaCl concentrations in sweat, and therefore approximate sweat rate. Changes in GSR correlate linearly with NaCl concentrations, where 20 μS corresponds to 35 mMol of NaCl, and 60 μS corresponds to 63 mMol. Once the sweat sensing device derived a NaCl concentration, it could then use a lookup table to determine sweat rate. However, GSR changes may vary considerably from individual to individual in relation to changes in sweat NaCl content, therefore using GSR change alone would only provide a very rough estimate of sweat rate.

Similarly, in other embodiments, sweat conductivity can be measured and used to determine Cl− content, which can be used as a proxy for sweat rate. Sweat conductivity is mainly attributable to sweat's Cl− content, since Cl− ions represent the vast majority of negative ions in sweat. As with other methods described herein, once the device determined a sweat Cl− concentration, it could then use a lookup table to determine sweat rate.

In other embodiments, GSR or sweat conductivity readings can be used in conjunction with contemporaneous ion selective electrode sensor (“ISE”)-derived sweat ion measurements to provide improved calculations of sweat rate. Comparisons of GSR/sweat conductivity and measurements of ISEs may need to be adjusted to account for sensor response lag by the ISEs to ensure truly contemporaneous measurements are being compared. In addition, because GSR shows broad individual variability, correlating GSR with sweat Na+ or Cl− concentrations would benefit from some form of calibration. For example, when a sweat sensing device is first activated and taking measurements on a wearer, the device may compare GSR changes and Na+ concentrations throughout the three GSR sweating regimes. The device could then correlate Na+ concentrations to GSR readings for each regime, and by extension, a calculated sweat rate. Then, during subsequent sweating cycles, the device could measure GSR change and calculate a sweat rate based on the Na+ value correlated to that GSR measurement in the appropriate sweat regime. In other embodiments, calibration of GSR change to Na+ concentration could be accomplished beforehand by aggregating data for a particular individual, a particular phenotype, a fitness level, an age range, or other relevant characteristic. The correlated aggregated data may then inform a subsequent use of a sweat sensing device to improve sweat rate calculations for a wearer.

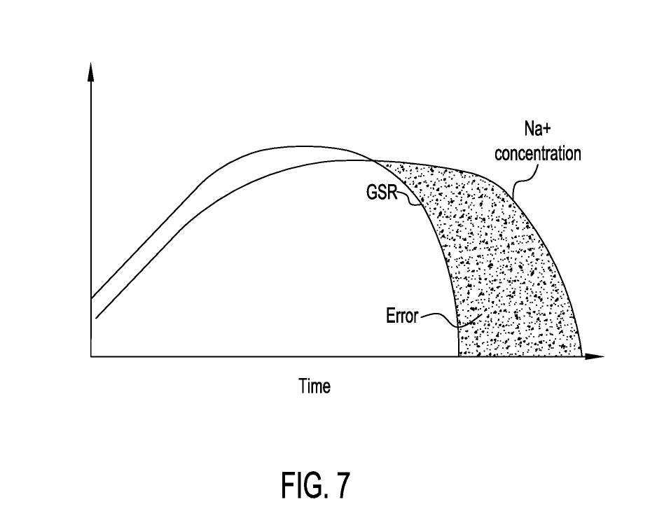

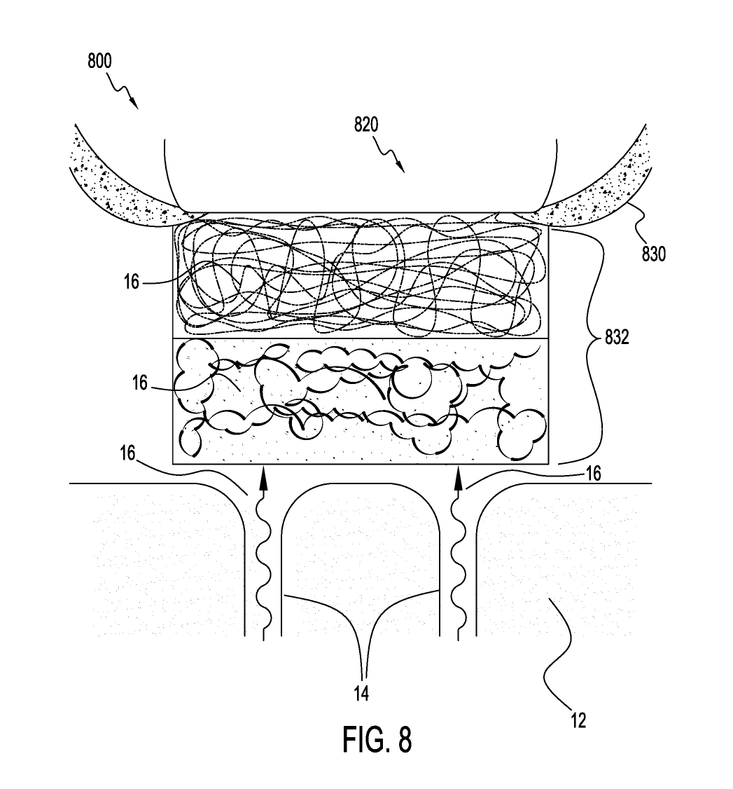

GSR or sweat conductivity changes may also be used to improve sweat rate and sweat ion loss calculations. For an example using GSR, FIG. 7 depicts a sweating cycle, in which sweat begins, progresses to the linear regime, and then declines. GSR change measurements track Na+ concentration during the portion of the cycle where sweat rate is increasing, but will lead Na+ while sweat rate is decreasing. This is attributable not only to the conductive properties of the sweat (which increase and decrease linearly with Na+ concentration), but also to the mechanics of the sweat sensing device as it collects and measures a sweat sample. During active sweating, sweat leaves the sweat gland and collects in a sweat volume located between the skin and the Na+ ISE sensor. When the sweat volume is filled, and a Na+ concentration measurement taken, the sweat sample is regarded as consisting entirely of new sweat. With reference to FIG. 8, a sweat sensing device 800 is placed on skin 12. During device operation, new sweat 16 exits the sweat glands 14 and enters the sweat volume 832. As new sweat 16 continues to enter the sweat volume 832, but before the older sweat 18 is entirely wicked away by microfluidic component(s) 830 and is displaced by new sweat 16, the sweat sample will consist of a mix of older sweat and newer sweat. The time required to fully refresh the sweat volume with new sweat is called the sample refresh time, and is inversely proportional to a chronologically assured sweat sampling rate (i.e., a sampling rate that measures analyte concentrations when the sweat volume is fully refreshed with new sweat). If the Na+ ISE 820 takes measurements at a sampling rate that is faster than the chronologically assured sampling rate, the Na+ concentration measurement will not reflect new sweat concentration, but rather an average of the new and old sweat concentration, which is effectively a physical integration of of the sweat sample over time.

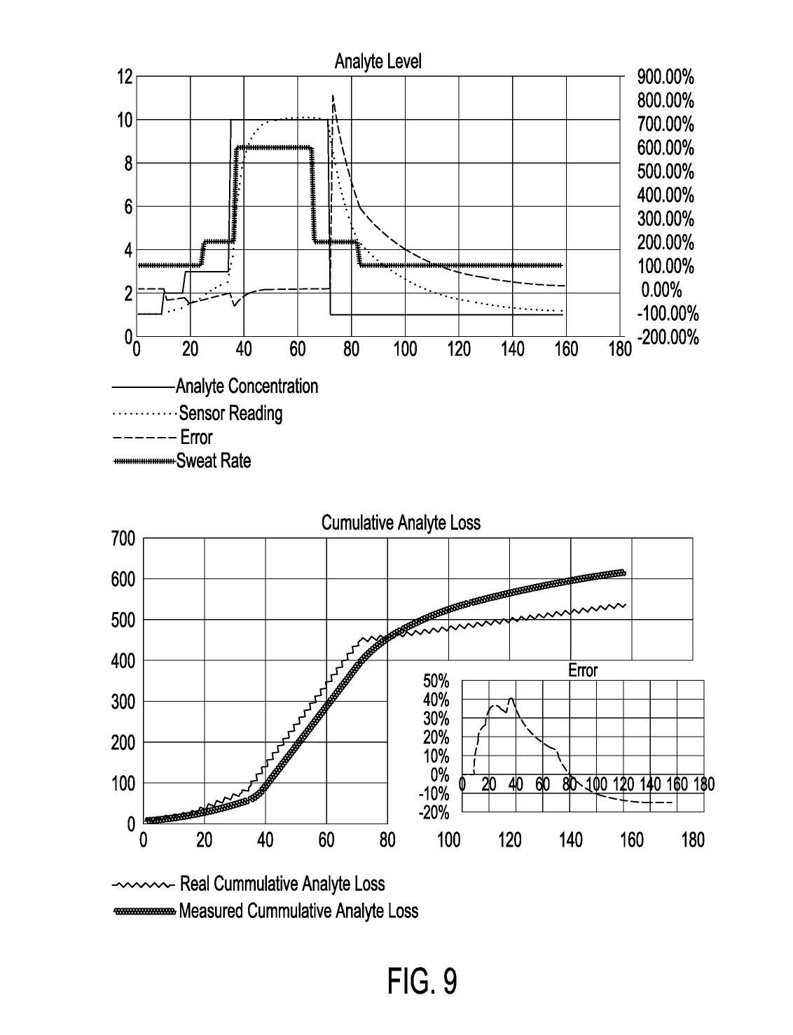

While sweat rate is increasing or if sweat rates are high, mixing of old and new sweat will be more rapid, and the sample refresh time will be relatively shorter, so chronologically assured sampling rates will be faster, allowing sensor measurements (sweat volume average concentration) to track actual concentration more accurately. Once sweat rate begins to decrease, however, GSR change will quickly reflect lower skin/sweat gland conductivity, but Na+ concentration readings will remain elevated because new sweat will not displace older sweat as quickly. If a sweat sensing device were to continue to sample Na+ concentrations at rates above the chronologically assured sampling rate, and then calculate sweat rate and Na+ concentration based on these lagging readings, many sweat sensing device functions would suffer in accuracy. For example, estimates of total water loss or electrolyte loss can be significantly overestimated, especially for sweat sensing applications where the wearer experiences multiple sweating cycles. See, e.g., FIG. 9.

During sweat sensing device operation, therefore, if GSR and Na+ readings indicate the initiation of a sweat cycle, a subsequent GSR change indicating a decrease in skin conductivity is used to indicate the timing of sweat rate decrease. The GSR change during this portion of the sweat cycle may then used to approximate Na+ concentration, as well as sweat rate. The device can also use GSR change to slow sweat sampling rates to improve chronological assurance. Lagging Na+ concentration measurements can also be discarded or corrected to account for the decreasing sweat rate.

To correct Na+ concentration readings during the declining portion of a sweat cycle, the sweat sensing device can employ a function that models how the mixing of old and new sweat affects Na+ sensor readings. With such a model, the device may more accurately interpret its measurements to reflect new sweat concentration. As a first-order correction, the device may model the sweat sample as a fixed volume with new sweat added and old sweat displaced. Each volume of new sweat displaces an equal volume of old sweat from the sweat sample. The model also assumes instant mixing of new sweat with old sweat in each time period dt. With a known sweat volume and accurate sweat rate measurement that is independent of Na+ and Cl− concentration, e.g., GSR, sweat conductivity, or micro-thermal flow sensors, the model can accurately calculate the area under the Na+ curve that is due to old sweat, which may then be subtracted from the Na+ readings to provide the new sweat Na+ concentration during decreasing sweat rates. The model may be improved by accounting for sensor response lag. Because ISE sensors generally respond more quickly to increases in analyte concentration than to decreases, sensor response lag will tend to exacerbate the problem presented by sweat sample mixing. The model may also be improved by including a mixing function that would account for the sweat volume's physical geometry, sweat rate and or wicking rate.

The integration technique disclosed herein may also be used to determine the concentration of other analytes, or the net loss of such analytes during periods of declining sweat rates. Because Cl− behaves in a similar way to Na+ in sweat, a similar process is applied to Cl− ISE measurements. ISE-derived Cl− concentrations therefore could be used with GSR or sweat conductivity to calculate sweat rate, or such sweat rate calculations could be used to corroborate or improve sweat rate calculations based on GSR and Na+ measurements, or sweat conductivity measurements. GSR or sweat conductivity changes indicating decreases in sweat rate could similarly be used to correct ISE-derived Cl− concentration measurements or sweat rate calculations based on Cl−, during periods of decreasing sweat rate.

Similarly, GSR or sweat conductivity changes could be compared with contemporaneous K+ measurements to improve determinations of the wearer's sweat regime. For example, an increasing GSR reading accompanied by a high K+ concentration (around 9 mMol) would indicate a very low, or negligible sweat rate. Likewise, a steady GSR reading accompanied by a declining K+ concentration would indicate a low sweat rate (below 0.4 μL/cm²/min). A linearly increasing GSR measurement accompanied by a steady K+ measurement (around 6 mMol) would indicate active sweating in excess of 0.4 μL/cm²/min.

The sweat sensing device may deploy the different modules, such as the Sweat Rate Module just described, in an iterative manner. For example, Hardware Calibrated values may inform a filtering routine for Na+ and K+. These filtered values may then receive first order Bio Calibration based on detected pH and sensor temperature readings. The Sweat Rate Physiological Module may then use these calibrated values to determine a calculated sweat rate value. The sweat rate value may then inform the device to adjust sweat sampling rate to ensure chronologically valid data for subsequent Na+ and K+ measurements, or to refine pH, temperature, or other first order calibration input. The device could then reprocess the original filtered Na+ and K+ values through the Bio Calibration routine using the refined pH value. The refined Na+ and K+ values may then inform a refined calculated sweat rate value.

The device may use data filtering results and sweat rate calculations to inform device control. As mentioned above, sweat rate can inform the device as to chronologically assured sweat sampling rates, and the device can adjust sensor activation accordingly. Sweat rate values may also prompt the device to discard sensor data, for example if the sweat rate were too low or too high, or to adjust sweat stimulation activity to tune a sweat rate for a particular sensor application. If a sensor produces data that gets filtered, the device may deactivate the sensor as malfunctioning, or may adjust the sensor's power levels, order a cleaning cycle, divert power to another sensor with the same function, or other necessary adjustments.

Once the device has sufficiently calibrated and refined the analyte concentration values, the algorithm then routes the data for further use. The algorithm my send data to a commercial health information aggregation application, such as Apple's HealthKit™, Google Fit™, Nike+ Training Club™, and so on. The algorithm may also provide data to a Primary Application, which is comprised of additional Physiological Modules, for example modules for developing outputs describing dehydration, stress level, muscle fatigue, or other physiologically meaningful data outputs. The refined output data will also be archived at this stage for quality control purposes.

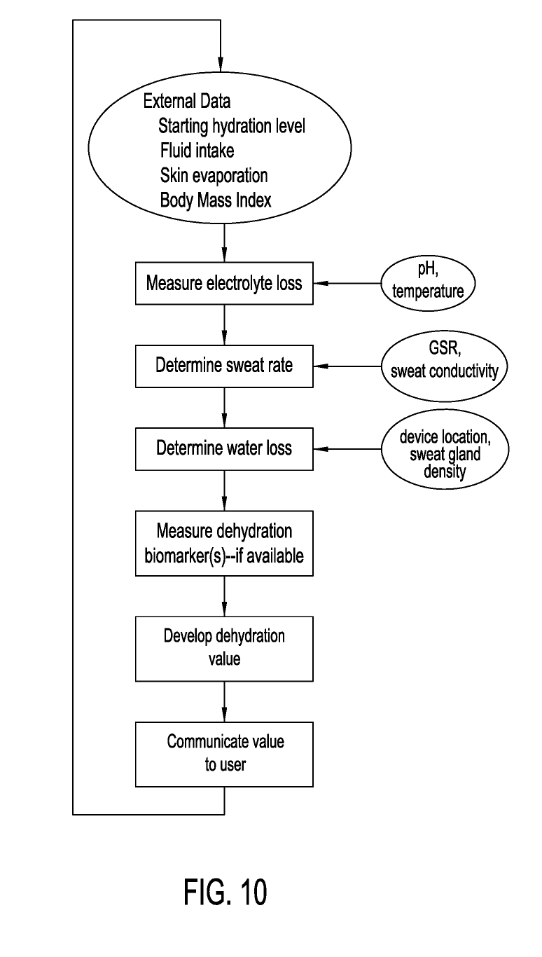

With reference to FIG. 10, in one such embodiment of the invention, the sweat sensing device could be configured and used to perform a Physiological Module to indicate whether an individual is becoming dehydrated. Dehydration (hypohydration) is the excessive loss of body water, with an accompanying disruption of metabolic processes. Dehydration actually refers to one of two conditions, which include hypernatremia, the loss of body water and attendant increase in NaCl and other solute concentrations; and hypovolemia, the loss of blood volume. Humans most commonly experience a form of hypovolemia in which both water volume and solute levels are depleted. Dehydration symptoms generally become noticeable after 2% of body water volume has been lost, but a decrease in body water volume of up to 3% to 4% is not usually problematic. However, dehydration symptoms become more severe as water loss continues. Decreased plasma volume causes an increase in heart rate and respiration rate, while decreased sweating causes body temperature to rise. At around 5% to 6% water loss, dehydration symptoms may include lethargy, sleepiness, headaches, nausea, and tingling in the limbs. At a 10% to 15% fluid loss, muscle spasms, shriveled skin, dimmed vision, reduced or painful urination, and delirium will present as symptoms. Body water losses over 15% are usually fatal as the kidneys and other organs fail. See Kleiner, S. M. (February 1999), “Water: an essential but overlooked nutrient,” Journal of the American Dietetic Association 99 (2): 200-6; Ashcroft, F., “Life Without Water in Life at the Extremes,” Berkeley and Los Angeles, 2000, 134-138. It is therefore useful for a variety of purposes to detect dehydration levels and trends before symptoms become a problem for an individual. A sweat sensing device configured to determine a user's dehydration state could take at least three main approaches, which could be used alone or in conjunction with at least one other approach to improve the device's predictive capability.

One approach involves the use of a sweat sensing device to detect or measure a biomarker or set of biomarkers that indicate an individual has become, or is becoming dehydrated. One candidate biomarker is the urea nitrogen (“BUN”) to creatinine ratio in blood. Urea and creatinine are nitrogenous end products of metabolism. Both are relatively small molecules that distribute throughout total body water. See Agrawal, M., Swartz, R. (April 2000), “Acute Renal Failure,” Am. Fam. Physician, 61(7): 2077-88. When an individual becomes dehydrated, water in the bloodstream decreases and urea is reabsorbed into the blood, which causes the BUN level to rise. If the dehydration state continues for a prolonged period, creatinine levels in the blood will also eventually rise, but do so at a lower rate than the BUN increase. Therefore, dehydration causes an increase in the BUN to creatinine ratio in the blood. Under normal hydration conditions, the BUN to creatinine ratio is ˜10:1. If the BUN to creatinine ratio exceeds 20:1, an individual is likely in a dehydrated state.

A similar ratio of analytes in sweat may also indicate the level of dehydration by correlating the sweat readings with the BUN to creatinine ratio. Sweat levels of urea, uric acid, and creatinine have been studied in relation to their concentrations in blood. Chen-Tsai Huang, et al., “Uric Acid and Urea in Human Sweat,” Chinese Journal of Physiology 45(3): 109-115, 2002; Yassar Y. Al-Tamar, Emam A. Hadi, Imad eldin I. Al-Badrani, “Sweat Urea, Uric Acid and Creatinine Concentrations in Uraemic Patients,” Urological Research, 25(5): 337-40, 1997 (showing blood serum creatinine levels of 0.286 mMol/L corresponding to a sweat concentration of 0.08 mMol/L).

Other promising candidates for dehydration biomarkers include cortisol, vasopressin, lactate, renin, angiotensin, and aldosterone, or one of their metabolites. A sweat sensing device could be configured with a plurality of sensors to measure concentrations, ratios, trends, or merely the presence of such potential biomarkers. Other common sweat analytes, such as Na+, K+, Cl−, and NH4+, may also prove useful in developing a profile indicative of dehydration.

The sweat concentration of urea in relation to blood serum levels has also been shown to vary with sweat rate, external temperature, patient age, kidney function, activity level, and other factors. Translation of sweat sensor readings into a meaningful assessment of dehydration therefore also may require certain information about the individual, and physiological knowledge available to those skilled in the art. The sweat sensing device would account for such factors using interpretive algorithms.

Solid-state urea biosensors consisting of a tin oxide (SnO₂) pH electrode, a nonactin-based ammonium ion-selective electrode (ISE), and an ammonium ion-selective membrane, have been demonstrated that respond rapidly, stably and linearly in the range of blood urea concentrations between 0.013 mM and 10 mM. See Nien Hsuan Chou, Jung Chuan Chou, Tai Ping Sun, Shen Kan Hsiung, “Differential type solid-state urea biosensors based on ion-selective electrodes,” Sensors and Actuators B, 130 (2008) 359-366. Creatinine is also a reliably detectable molecule by using amperometric biosensors. Creatinine can be detected through the use of electrochemical enzymatic biosensors based on carbon rod electrodes modified with a mixture of sarcosine oxidase and creatine amidinohydrolase. Such sensors can demonstrate a linear response up to 4 mM sarcosine and up to 3.5 mM creatinine. See Ramanavicius, A. “Amperometric biosensor for the determination of creatine,” Analytic Bioanalytic Chemistry, (2007) 387:1899-1906. Since these analytes are detectable in blood, sensor technology tailored for use in sweat is accessible to those skilled in the art.

A second approach involves an estimation of net fluid loss experienced by a wearer during use of a sweat sensing device. A sweat sensing device configured to estimate net fluid loss would require a number of basic components, including sweat analyte sensors to detect Na+, Cl−, and K+, and a reference electrode, and may also include other sensors, such as a GSR sensor, a pH sensor, a micro-thermal flow rate sensor, a sweat conductivity sensor, or a temperature sensor. The device would also include the capability to perform data archiving, data normalization, hardware calibration and bio calibration. The device may also track outside information, such as a wearer's initial hydration state (e.g., well-hydrated, thirsty, etc.), activity level, their fluid intake, environmental humidity, air temperature, or other relevant factors to provide inputs for the dehydration state calculation.

The wearer's hydration state may be determined during sweat sensing device operation by estimating total body fluid loss by measuring sweat rate, and comparing the estimated fluid loss to starting hydration state and fluid intake during device operation. The sweat sensing device measures sweat rate during device operation by using a plurality of micro-thermal flow sensors, GSR measurements of skin conductivity, sweat conductivity, electrolyte (Na+, Cl−) concentration levels, or a combination of these means. The device will also need inputs about the anatomical location of the sweat sensing device on the body, since different areas of the body experience different sweat rates. The device will also need information about the physical characteristics of the wearer, such as Body Mass Index, height, or weight, in order to calculate a body surface area for the wearer. With the foregoing information, the device can calculate a fluid loss rate for the wearer. This fluid loss rate may also account for total evaporative water loss, sweat reduction caused by the patch itself, or other factors relevant to improve the calculated value. Integrating the fluid loss rate calculations over the course of device operation provides a calculated cumulative fluid loss. Combined with the wearer's water intake during device operation., the device may then determine water loss as a function of body mass. This calculated body water volume loss percentage (between 0% and 15%) represents the wearer's dehydration status.

A third approach would be to measure the amount of sweat collected by a sweat sensing device, and extrapolate total fluid loss based on the measured volume. In such embodiments, the device may include a sweat collection reservoir and a means of estimating the volume of sweat collected in the reservoir, such as an impedance electrode.

Using correlated aggregated sweat sensor data, including information about a specific device wearer—or generally about other relevant factors, such as temperature, or humidity—the individual's dehydration profile value can be developed, which is communicated to the sweat sensing device. The dehydration state would reflect, with reasonable accuracy, whether a person was dehydrated, and the severity of the dehydration under the particular circumstances experienced by the patch wearer.

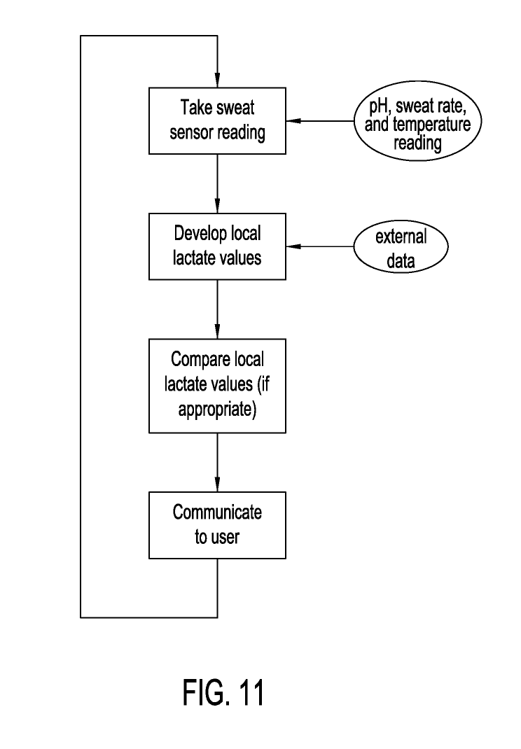

With reference to FIG. 11, in another disclosed embodiment, a sweat sensing device can be used to determine a local sweat lactate concentration. Lactate is used by muscle groups during periods of intense or prolonged exertion. Muscle tissue (and eccrine sweat glands) use lactate to sustain the purine nucleotide cycle (PNC) and tricarboxylic acid cycle (Krebs cycle), which together allow energy production in the absence of adequate oxygen. During such periods of intense exertion, glucose is oxidized into pyruvate, which is then turned into lactate as part of the oxidation of NADH to NAD+. Lactate increases in blood may therefore be the result of three different causes: (1) more lactate is being produced; (2) the muscles are not using or removing as much lactate; or (3) lactate is being produced at a greater rate than it is being used. When an individual is at rest, blood lactate concentrations are usually around 1-2 mMol/L. Under intense exertion, blood lactate levels may reach about 20 mMol/L, peaking at about 3-8 minutes after exercise. Lactate can then drift upward to 25 mMol/L in the post-exertion period, and may remain elevated for an hour or more. Goodwin, M., et al., “Blood lactate measurements and analysis during exercise,” J. Diabetes Science & Tech. Vol. 1, Issue 4, July 2007. There are three blood lactate metrics that may inform an individual's physical performance. The lactate threshold (LT) is the point after which lactate begins to accumulate in the blood at exponential rates. Further along the spectrum of intense exercise is the onset of blood lactate accumulation (OBLA) value, which represents a point where the lactate accumulation rate begins to accelerate. Another metric is the maximal lactate steady state (MLSS), which is the most intense sustained exercise level that can be maintained while maintaining a constant blood lactate level. Goodwin, M., et al.

The translation of sweat lactate concentrations to those in blood is complex and not well understood. For example, at higher sweat rates, the metabolic activity of sweat glands produces lactate amounts which exceed lactate diffusion into sweat from blood. During intense exercise, therefore, sweat measurements of lactate may show a spike in concentration that corresponds to the exercise intensity, but that does not correlate to blood lactate concentration. As a result, sweat measurements of lactate that correlate with blood concentrations may need to be made a very low sweat rates (where sweat lactate will be primarily from diffusion rather than gland production), or a proxy analyte or analytes may be required. Sonner, Z., et al., “The microfluidics of the eccrine sweat gland, including biomarker partitioning, transport, and biosensing implications,” Biomicrofluidics 9, 031301 (2015); doi: 10.1063/1.4921039.

Because of this difficulty in translating sweat lactate to baseline blood concentrations, a preferred embodiment of the disclosed invention may be configured to detect local lactate production by discrete muscle groups. For example, a plurality of sweat sensing devices configured with lactate sensors could be placed on the skin in proximity to various muscle groups, i.e., quadriceps, calves, gluteals, pectorals, etc. The placement of the devices would enable the detection of local lactate spikes over those muscle groups experiencing intense exertion. Intense exertion can be determined by correlation to a blood lactate metric, such as OBLA, or other means. The devices may communicate to a network to inform the user which muscle groups are experiencing exertion, the duration and intensity of the exertion, or the relative intensity of exertion among muscle groups, e.g., left leg quadriceps versus right leg quadriceps. In addition, the devices may detect muscle fatigue, or impending fatigue. Alternatively, the devices may also be very low cost, with minimal onboard processing and communication components. For such embodiments, the devices could be configured to detect a certain threshold lactate concentration corresponding to intense exertion, fatigue or impending fatigue, or correlated to a known blood lactate metric. Outputs may be in the form of an LED light indication, a vibration signal, or an auditory alert from individual patch devices. Sensor technology known in the art for lactate includes aptamer-based sensors (EAB sensors), amperometric sensors, and optical sensors.

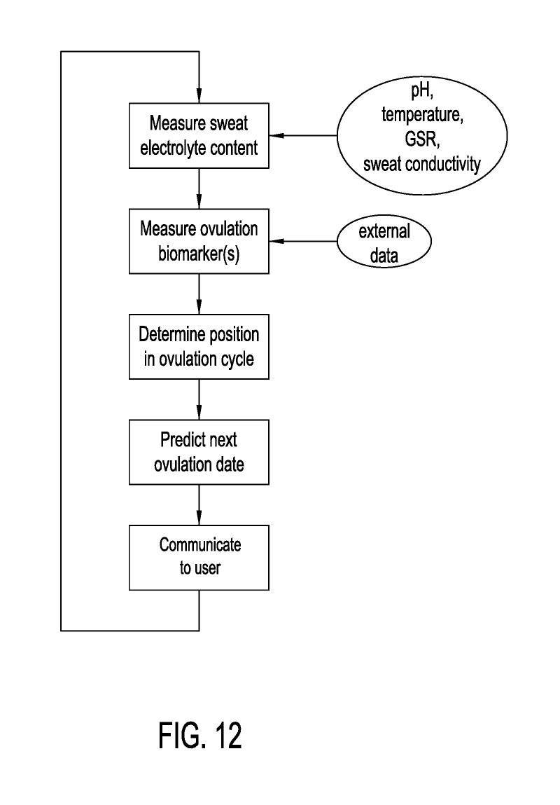

With reference to FIG. 12, in another embodiment of the invention, the sweat sensing device could be configured and used to indicate a human female individual's fertile stage in her ovulation cycle in order to improve opportunity for conception. For conception to occur, egg and sperm cells must be co-present in the uterus. During a typical 28-day ovulation cycle, an egg is present in the uterus for less than 24 hours, while a sperm can typically survive in the uterus for up to four days. To optimize fertility, therefore, it is necessary accurately to predict ovulation four days in advance. Ovulation activity in humans is triggered and regulated by the release of a number of hormones, including follicle stimulating hormone (FSH), luteinizing hormone (LH), estrogen (estradiol) and progesterone. Estradiol is known to begin an accelerated increase an average of five days prior to ovulation, with a peak occurring approximately one day before ovulation followed by a sharp decline. LH is perhaps the best indicator of impending ovulation, since it stays at low levels during most of the cycle, and then begins a very sharp increase about three days before ovulation, hitting a peak and immediate sharp decline at around 12 to 24 hours before ovulation. FSH also has a distinctive profile during the cycle, starting a gentle decrease about 10 days before ovulation, reaching a nadir and beginning sharp increase around three days prior to ovulation. FSH peaks at ovulation and begins a steep decline to a second nadir at around 10 days past ovulation. Progesterone maintains a low level in the female body during most of the ovulation cycle, with a sharp increase occurring about two days prior to ovulation on average. Progesterone builds to a peak by six or seven days after ovulation, and then begins to decrease if fertilization does not occur, thereby triggering the start of a new ovulation cycle. These hormones may manifest and be detected in sweat directly, or indirectly, by measuring changes in other detectable analyte concentrations. For example, estrogen receptor beta (ER β) molecules are found in eccrine sweat glands, as are progesterone receptors and LH receptors. Alternatively, a sweat sensing device may be used to detect a plurality of analytes to determine when to test for LH, which could then be accomplished using a conventional ovulation test. In other embodiments, the device could be configured to detect human chorionic gonadotropic hormone (hCG), which is produced by the embryo after implantation in the uterus, thereby indicating that pregnancy has occurred. In order to distinguish hCG produced by the embryo from LH, or hCG produced by the mother, the device may employ a β-hCG sensor, or other suitable sweat analyte suitable to indicate pregnancy.

In addition, these hormones cause changes in certain measurable characteristics of the female body that can be measured by a sweat sensing device, such as skin temperature, sweat onset relative to skin temperature, and sweat pH. There is also some evidence that sweat concentrations of Cl−, Na+, NH₄+, and other analytes may change in response to ovulation cycle hormonal changes. While many sources in the art have noted a Na+/C1− surge occurring in sweat measurements about 5 days prior to ovulation, much of this change is attributable to an increase in sweat rate, rather than a change in sweat concentration of these analytes. Even so, the method described herein may use such information if the sweat sensor is configured to distinguish sweat rate changes caused by ovulation from sweat rate changes caused by other factors, such as environment, stress, exercise, etc. Finally, the hormones secreted by the body prior to and during ovulation cause a number of other physical changes in the individual's body that could be considered as well, including changes in cervical mucus viscosity, cervical position, an increase in basal body temperature, and ovulation-related pains. These factors can be monitored in conjunction with sweat sensor-derived data to improve the predictive capability of the disclosed device and method.

The person's ovulation-related sweat data may be calculated to develop an ovulation state value. By comparing sweat concentrations and ratios of relevant analytes, the ovulation state would reflect, with reasonable accuracy, whether a person was likely in the fertile stage of her ovulation cycle, i.e., whether she is likely to ovulate within the next four to five days. The ovulation state value could also be used to generally predict when the fertile stage would occur by determining the person's current stage in her ovulation cycle.

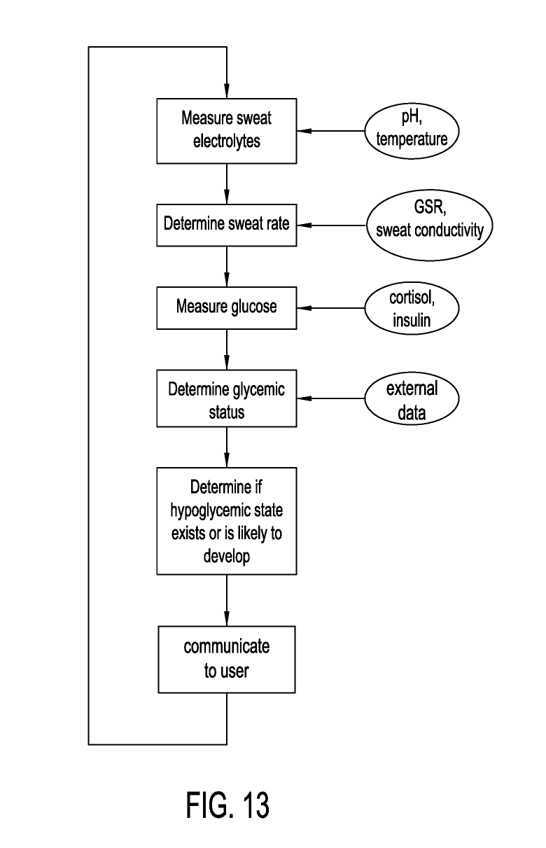

With reference to FIG. 13, in another embodiment of the invention, the sweat sensing device is configured and used to indicate that an individual had entered a hypoglycemic state, or a state of severely low blood glucose. Hypoglycemia is most commonly seen in adults and children with type 1 diabetes mellitus, but non-diabetics may also experience hypoglycemia. Because the brain and central nervous system require continuous fuel in the form of glucose to function, hypoglycemia manifests by increasing cognitive and nervous system dysfunction. When blood glucose falls below 3.6 mM, cognitive impairment will begin to present. At 2.2 mM, sweating will commence and cognitive impairment will become obvious to the outside observer. Below this level, the individual may experience seizures, and below 0.55 mM, the individual may enter a diabetic coma.

Accuracy in detecting hypoglycemia is critically important, both due to the potential ramifications of the failure to correctly identify a hypoglycemic incident in progress, but also due to the high potential for false-positive interpretations of analyte levels. Because of these difficulties, developing a suitable hypoglycemia monitor has proven a particular challenge for practitioners in the art. Given this difficulty, the sweat sensor should be configured to comprehensively monitor the individual, both in terms of sweat analyte contents, but also in terms of other physical manifestations such as sweating or seizure onset. In addition to measuring sweat electrolytes such as Na+, Cl−, and K+, the sweat sensor may also be configured to detect glucose, insulin and cortisol levels using amperometric or aptamer-based biosensors. To further monitor the onset of hypoglycemia, the sweat sensor may also be configured with an accelerometer to detect seizure or coma, an impedance apparatus to measure galvanic skin response, as well as sweat pH, sweat rate and body temperature measurements, in addition to other relevant measurements. Together, these measurements are analyzed over time and in relation to each other to calculate a hypoglycemic profile value for the individual.

While hypoglycemia usually results from excessive insulin production in the body, there are a number of causes that are linked to the individual's age, previous incidents of hypoglycemia, nutrition, time of day, and organ function. Such information can be correlated with an individual's sweat sensor data to further enhance the monitoring of their hypoglycemic state. The wearer's glucose trend could be determined by measuring sweat glucose concentrations, and comparing those readings over time, e.g., over a 1 hour period.

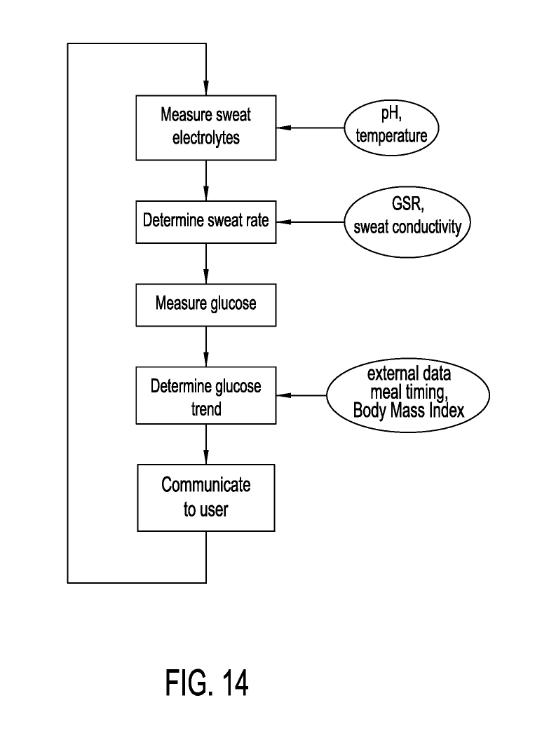

With reference to FIG. 14, another embodiment disclosed herein is a sweat sensing device capable of performing a glucose trend assessment. Such a device would require a number of basic components, including sweat analyte sensors to detect Cl− and K+, a reference electrode, a pH sensor, and a temperature sensor. The device would also include the capability to perform data archiving, data normalization, hardware calibration and bio calibration. The device may also track outside information, such as a user's activity level, their food intake, information from glucose monitors, or other relevant factors to provide input for the glucose trend calculation.

The device would be configured with a plurality of sensors to measure concentrations of sweat glucose. Sensor technology known in the art includes aptamer-based sensors (EAB sensors), or amperometric sensors. The glucose trend device would collect and interpret data relevant to the wearer's glucose level, and report significant changes to the wearer. For example, the glucose trend device measures a steady level of glucose in sweat over a period of 2 hours. At 2 hours 10 minutes into wearing the device, glucose sensors detect a sharp increase in sweat concentration. The glucose trend device then informs the user that a sudden increase in glucose (or a glucose spike) has occurred. Similarly, the device could interpret and report a sharp decrease in sweat molar concentration. Applications for such a device include dietary monitoring for improving health or human performance. For example, the interval between food intake and a glucose spike may provide information about the nutritional value of certain foods, such as the prevalence of rapidly metabolized carbohydrates or sugars. Similarly, the slope of a glucose spike may also prove informative of food nutritional content. Sharp decreases in sweat glucose, when correlated with physical activity, may inform performance capacity or timing of food intake to optimize performance.

In another embodiment of the invention, the sweat sensing device could be configured and used to indicate whether an individual was experiencing hypokalemia or hyperkalemia. These conditions are characterized by the body's inability to regulate blood K+ levels. Sweat levels of K+ tend to vary directly with blood concentrations of K+, therefore, consistent detection of abnormally high or abnormally low levels of K+ in sweat could indicate hyperkalemia or hypokalemia, respectively. In blood serum, K+ levels between 3.5 and 5.0 mEq/L are considered normal, while blood levels greater than 5.0 mEq/L indicate hyperkalemia, and levels less than 3.5 mEq/L indicate hypokalemia.

The states of hypo/hyperkalemia may have several different causes, which may also be indicated by a properly configured sweat sensor. For example, hypokalemia might be caused by low blood fluid volume (hypovolemia), diuretic medication, low magnesium levels, high blood pH, or high cortisol levels (stress), among other causes. A sweat sensor configured to detect dehydration, for example, could indicate a hypovolemic cause of hypokalemia. Hyperkalemia might be caused by deficient kidney function, rhabdomyolysis, steroid/aldosterone deficiency, low insulin, or digoxin toxicity, among others. A sweat sensor configured to detect sweat urea, for example could point to kidney function deficiency as the cause.

In another embodiment of the invention, the sweat sensor could be configured and used to indicate whether an individual had been exposed to a toxic exogenous substance. This type of screening test could be employed in a hospital waiting room, at the workplace, or other suitable location. Sensitivity of sensors could be low, as long as the screening test reliably showed the presence of the target substance. For example, a sensor could be configured with an ion selective electrode capable of detecting the presence of lead. The sweat sensing device could be enabled through algorithms to detect a threshold level, the toxic threshold, of lead in sweat that corresponds to the presence of lead in the bloodstream that indicates further medical investigation is merited. Other metals and heavy metals that might be detected include arsenic, beryllium, cadmium, chromium (VI), cobalt, lithium, mercury, polonium, thallium, and zinc. Other potential substances that could be detected as described herein include isocyanate; acrylamide; cotinine; methyl ethyl ketone; polybrominated biphenyl (PBB); N,N-Diethyl-meta-toluamide (DEET); 4-(methylnitrosamino)-1-(3-pyridyl)-1-butanol; perchlorate; fungicides; herbicides; insecticides; polycyclic aromatic hydrocarbons (PAH); polybrominated diphenyl ethers (PBDEs); dioxin-like chemicals; non-dioxin-like polychlorinated biphenyls (PCBs); parabens; perflourochemicals (PFCs); phthalates; trihalomethanes; volatile organic compounds and their metabolites (VOC); and environmental phenols such as benzophenone-3, bisphenol-A (BPA), triclosan, and 4-tert-octylphenol.

Alternatively, rather than detect the toxic substance directly, a sweat sensing device can be configured to detect a tracer molecule that is combined with the toxic substance. For example, an insecticide or fuel product may prove difficult to detect with a sweat sensing device. In such cases, an easily detectable tracer could be added to the toxic substance to enable detection.

This has been a description of the present invention along with a preferred method of practicing the present invention, however the invention itself should only be defined by the appended claims.

1. A method of using a sweat sensing device to determine an individual's dehydration state, comprising:
taking at least one sweat analyte measurement, where the analyte is a biomarker indicative of the individual's dehydration state;
developing a dehydration state value for the individual; and
communicating said value to a device user.

2. The method of claim 1 where the device takes at least one measurement of sweat pH.

3. The method of claim 1, where the analyte is at least one of the following: Na+, K+, Cl−, and NH₄+.

4. The method of claim 1, further including measuring a sweat rate value using at least one of the following: a GSR measurement, a thermal flow rate measurement, a collected sweat volume measurement, and a sweat conductivity measurement.

5. The method of claim 1, where the analyte is at least one of the following biomarkers: urea, creatinine, cortisol, vasopressin, lactate, renin, angiotensin, aldosterone, and a metabolite of the biomarker.

6. The method of claim 1, where said dehydration state value is developed using aggregated sweat sensor data that is correlated to relevant external information.

7. The method of claim 6, where the external information includes at least one of the following: air temperature, humidity, the individual's age, the individual's initial hydration state, the individual's fluid intake, the individual's body mass index, the individual's kidney health, the individual's fitness level, the individual's sex, the individual's heart rate, and the individual's recent physical activity.

8. A method of using a sweat sensing device to determine an individual's dehydration state, comprising:
taking at least one sweat analyte measurement;
using the at least one analyte measurement to develop a sweat rate;
taking at least one measurement of sweat rate independent of the developed sweat rate;
using at least one of the developed sweat rate and measured sweat rate to determine the individual's total fluid loss;
determining the individual's starting hydration level;
determining the individual's fluid intake during device operation;
developing a dehydration state value for the individual; and
communicating said value to a device user.

9. The method of claim 8 where the device takes at least one measurement of sweat pH.

10. The method of claim 8, where the analyte is at least one of Na+, Cl−, K+, NH₄+ urea, creatinine, cortisol, vasopressin, lactate, renin, angiotensin, aldosterone, and a metabolite of the analyte.

11. The method of claim 10, where the developed sweat rate determination includes correlating the at least one analyte measurement to a sweat rate.

12. The method of claim 8 where the measured sweat rate is developed using at least one of the following: a GSR measurement, a thermal flow rate measurement, a collected sweat volume measurement, and a sweat conductivity measurement.

13. The method of claim 8, where the total fluid loss accounts for at least one of the following: a device location on the individual's body, a number of sweat glands under the device, an amount of evaporative water loss, and an amount of sweat suppressed by the device.

14. The method of claim 8, where the starting hydration level accounts for at least one of the following: air temperature, humidity, the individual's age, the individual's initial hydration state, the individual's fluid intake, the individual's body mass index, the individual's kidney health, the individual's fitness level, the individual's sex, the individual's heart rate, and the individual's recent physical activity.

15. The method of claim 8, where said dehydration state value is developed using aggregated sweat sensor data that is correlated to relevant external information.

16.-80. (canceled)

81. A method of correcting sweat sensing device measurements during periods of decreasing sweat rate, comprising:
having a known sweat volume;
taking at least one first sweat analyte measurement;
developing a first sweat rate value contemporaneous with the first analyte measurement;
taking at least one second sweat analyte measurement;
developing a second sweat rate value contemporaneous with the second analyte measurement;
correcting the second sweat analyte measurement if the second sweat rate value is less than the first sweat rate value, where the correction mathematically models the relative contribution of old sweat and new sweat in the sweat volume.

82. The method of claim 81, where the analyte is at least one of Na+, Cl−, K+, NH₄+ urea, creatinine, cortisol, vasopressin, lactate, renin, angiotensin, aldosterone, and a metabolite of the analyte.

83. The method of claim 81, where the sweat rate is based on one of the following: GSR, sweat conductivity, a micro-thermal flow meter, a collected sweat volume measurement, and electrolyte concentrations.

84. The method of claim 81, where the correction includes a mathematical model of the mixing rate of old and new sweat in the sweat volume.

85. The method of claim 81, where the correction accounts for sensor response lag.

86.-96. (canceled)
